(19) United States
(12) Patent Application Publication
(10) Pub. No.: US 2004/0136918 A1
(43) Pub. Date: Jul. 15, 2004

(54) Pharmaceutical formulations

(76) Inventors: Ronique Nichele Garrett, Durham, NC (US); Paul Johnson, Ware (GB); Anthony James Taylor, Ware (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/469,945

(22) PCT Filed: Mar. 4, 2002
(86) PCT No.: PCT/GB02/00909

(51) Int. Cl. A61L 9/04; A61K 9/14; A61K 31/573

(52) U.S. Cl. 424/46; 514/171

(57) ABSTRACT

The present invention is concerned with pharmaceutical formulations comprising a combination of R-salmeterol and flucticasone propionate and the use of such formulations in medicine, particularly in the prophylaxis and treatment of respiratory diseases.

The present invention is concerned with combinations of R-salmeterol and fluticasone propionate, pharmaceutical formulations containing a combination of R-salmeterol and fluticasone propionate and the use of such formulations in medicine, particularly in the prophylaxis and treatment of respiratory diseases.

Salmeterol is a potent, long lasting beta₂ adrenoreceptor-agonist commonly prescribed for the treatment of patients with obstructive airway disease such as asthma. Salmeterol is marketed as the racemate under the trademark SEREVENT™.

The R and S isomers of salmeterol are known. European patent application number EP0422889 and U.S. Pat. No. 5,919,827 both relate to the R-isomer of salmeterol and suggest it has a particularly advantageous profile of action. More particularly, U.S. Pat. No. 5,919,827 suggests that the use of the R-isomer for the treatment of inter alia asthma provides a safe and effective therapy while reducing undesirable side effects typically associated with beta-adrenergic drugs. However, International patent application number WO99/13867 suggests the converse, namely that it is the administration of the S-isomer of salmeterol which minimises undesirable side effects.

Fluticasone propionate is an anti-inflammatory corticosteroid, described in GB 2088877, and is systematically named S-fluoromethyl-6α,9α-difluoro-11β-hydroxy-16α-methyl-17α-propionyloxy-3-oxoandrosta-1,4-diene-17β-carbothioate. Fluticasone propionate is now used clinically for the treat of bronchial asthma and related disorders.

Although R-salmeterol and fluticasone propionate may be effective therapies, there exists a clinical need for asthma therapies having potent and selective action and having an advantageous profile of action.

Therefore, according to the present invention there is provided a combination of R-salmeterol or a physiologically acceptable salt or solvate thereof and fluticasone propionate or a physiologically acceptable salt or solvate thereof.

It is to be understood that the present invention covers all combinations of particular and preferred aspects of the invention described herein.

The formulations according to the invention employ the R-isomer substantially free of the S-isomer, by which is meant less than 10%, preferably less than 1% and especially less than 0.1% by weight of the S-isomer relative to the R-isomer.

Suitable salts according to the invention include those formed with both organic and inorganic acids. Physiologically acceptable acid addition salts include but are not limited to those formed from hydrochloric, hydrobromic, sulphuric, citric, tartaric, phosphoric, lactic, pyruvic, acetic, trifluoroacetic, succinic, oxalic, fumaric, maleic, oxaloacetic, methanesulphonic, ethanesulphonic, p-toluenesulphonic, benzenesulphonic, isethionic, and naphthalenecarboxylic, such as 1-hydroxy-2-naphthoic acids. R-salmeterol will preferably be in the form of its 1-hydroxy-2-naphthoate (xinafoate) salt.

It will be appreciated that the medicaments of the combination may be administered simultaneously, either in the same or different pharmaceutical formulations or sequentially. If there is sequential administration, the delay in administering the second medicament should not be such as to lose the beneficial therapeutic effect of the combination.

While it is possible for the medicaments of the combination to be administered as the raw chemical, it is preferable to present them as a pharmaceutical formulation. When the individual medicaments of the combination are administered separately, they are generally each presented as a pharmaceutical formulation as described previously in the art.

Pharmaceutical formulations are often prescribed to the patient in “patient packs” containing the whole course of treatment in a single package. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions and, therefore, lead generally to more successful treatment. It will be understood that the administration of the combination of the invention by means of a single patient pack, or patient packs of each component medicament, and containing a package insert instructing the patient to the correct use of the invention is a desirable additional feature of the invention.

According to a further aspect of the present invention, there is provided a pharmaceutical formulation comprising R-salmeterol or a physiologically acceptable salt or solvate thereof and fluticasone propionate or a physiologically acceptable salt or solvate thereof, and a pharmaceutically acceptable carrier or excipient, and optionally one or more other therapeutic ingredients. According to a preferred aspect of the present invention, there is provided a pharmaceutical formulation comprising R-salmeterol xinafoate and fluticasone propionate, and a pharmaceutically acceptable carrier or excipient, and optionally one or more other therapeutic ingredients. In the most preferred aspect, the above pharmaceutical formulations are suitable for administration by inhalation.

As mentioned above, both R-salmeterol and fluticasone propionate and their physiologically acceptable salts and solvates have been described for use in the treatment of respiratory disease. Therefore, formulations of R-salmeterol and fluticasone propionate and their physiologically acceptable salts and solvates, have use in the prophylaxis and treatment of clinical conditions for which a selective β₂-adrenoreceptor agonist and/or an anti-inflammatory corticosteroid is indicated. Such conditions include diseases associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, such as allergic and seasonal rhinitis).

Accordingly, the present invention provides a method for the prophylaxis or treatment of a clinical condition in a mammal, such as a human, for which a selective β₂-adrenoreceptor agonist and/or anti-inflammatory corticosteroid is indicated, which comprises administration of a therapeutically effective amount of a combination of R-salmeterol or a physiologically acceptable salt or solvate, thereof and fluticasone propionate or a physiologically acceptable salt or solvate thereof. The present invention further provides a method for the prophylaxis or treatment of a clinical condition in a mammal, such as a human, for which a selective β₂-adrenoreceptor agonist and/or anti-inflammatory corticosteroid is indicated, which comprises administration of a therapeutically effective amount of a pharmaceutical formulation comprising R-salmeterol or a physiologically acceptable salt or solvate thereof and fluticasone propionate or a physiologically acceptable salt or solvate thereof, and a pharmaceutically acceptable carrier or excipient. In a preferred aspect, there is provided such a method which comprises administration of a therapeutically effective amount of a pharmaceutical formulation comprising R-salmeterol xinafoate and fluticasone propionate, and a pharmaceutically acceptable carrier or excipient. In particular, the present invention provides such methods for the prophylaxis or treatment of a disease associated with reversible airways obstruction such as asthma, chronic obstructive pulmonary disease (COPD), respiratory tract infection or upper respiratory tract disease.

In the alternative, there is provided a combination of R-salmeterol or a physiologically acceptable salt or solvate thereof and fluticasone propionate or a physiologically acceptable salt or solvate thereof, for use in therapy, particularly for use in the prophylaxis or treatment of a clinical condition for which a selective β₂-adrenoreceptor agonist and/or anti-inflammatory corticosteroid is indicated. In particular, there is provided a pharmaceutical formulation comprising R-salmeterol or a physiologically acceptable salt or solvate thereof (suitably, R-salmeterol xinafoate) and fluticasone propionate or a physiologically acceptable salt or solvate thereof, and a pharmaceutically acceptable carrier or excipient for use in therapy, particularly for use in the prophylaxis or treatment of a clinical condition for which a selective β₂-adrenoreceptor agonist and/or anti-inflammatory corticosteroid is indicated. In a preferred aspect, the invention is concerned with the prophylaxis or treatment of a disease associated with reversible airways obstruction such as asthma, chronic obstructive pulmonary disease (COPD), respiratory tract infection or upper respiratory tract disease.

The amount of R-salmeterol and fluticasone propionate, or a physiologically acceptable salt or solvate thereof which is required to achieve a therapeutic effect will, of course, vary with the particular compound, the route of administration, the subject under treatment, and the particular disorder or disease being treated. As a monotherapy, U.S. Pat. No. 5,919,827 teaches that R-salmeterol may be generally administered to humans by inhalation at a dose of about 25 mcg to about 50 mcg, one or more times a day, whilst EP0422889 teaches a daily dose of 0.005 mg to 100 mg. As a monotherapy, fluticasone propionate is administered to adult humans by aerosol inhalation at a dose of from 100 mcg to 1000 mcg twice daily, preferably 200 mcg to 500 mcg. The dose of each component of the combination will in general be that employed for each component when used alone, though use of the combination of medicaments may allow for a lower dose of either one or both medicaments to be used. Typically, administration may be one or more times, for example from 1 to 8 times per day, giving for example 1,2,3 or 4 puffs each time.

Suitable daily doses by inhalation, may be, for example in the range 15 microgram to 10 mg, preferably 15 to 200 microgram of R-salmeterol, thus, for example, each valve actuation may deliver 10 to 500 microgram, preferably 25 to 200 microgram (and the dose can be delivered in either one or two actuations) and a dose of fluticasone propionate of 50 mcg to 1.0 mg, preferably 100 mcg to 500 mcg. Typically each filled canister for use in a metered dose inhaler contains sufficient suspension to deliver 30, 60, 120 or 200 metered doses or puffs of medicament. It is well known to a person skilled in the art that the canister may be overfilled with suspension equivalent to up to 40 puffs to ensure that the intended number of puffs can be delivered.

Preferred unit dosage formulations are those containing a pharmaceutically effective dose, as hereinbefore recited, or an appropriate fraction thereof, of the medicament. Thus, in the case of formulations designed for delivery by metered dose pressurised aerosols, one actuation of the aerosol may deliver half of the therapeutically effective amount such that two actuations are necessary to deliver the therapeutically effective dose.

The pharmaceutical formulations according to the invention may further include further therapeutic ingredients particularly those which are useful in inhalation therapy. Appropriately, one or more further therapeutic ingredients may thus be selected from for example, analgesics, e.g. codeine, dihydromorphine, ergotamine, fentanyl or morphine; anginal preparations, e.g. diltiazem; antiallergics, e.g. cromoglycate (e.g. as the sodium salt), ketotifen or nedocromil (e.g. as the sodium salt); antiinfectives e.g. cephalosporins, penicillins, streptomycin, sulphonamides, tetracyclines and pentamidine; antihistamines, e.g. methapyrilene; anti-inflammatories e.g. flunisolide, beclomethasone (preferably as the dipropionate ester), budesonide, ciclesonide, rofleponide, tipredane, triamcinolone (e.g. as the acetonide), mometasone (preferably as the furoate ester) or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g. noscapine; bronchodilators, e.g. albuterol (e.g. as free base or sulphate), ephedrine, adrenaline, fenoterol (e.g. as hydrobromide), formoterol (e.g. as fumarate), terbutaline (e.g. as sulphate), isoprenaline, metaproterenol, phenylephrine, phenylpropanolamine, pirbuterol (e.g. as acetate), reproterol (e.g. as hydrochloride), rimiterol, isoetharine, tulobuterol, orciprenaline, 4-hydroxy-7-[2-[[2-[[3-(2-phenylethoxy)propyl]sulfonyl]ethyl]-amino]ethyl-2(3H)-benzothiazolone or (−)-4-amino-3,5-dichloro-α-[[[6-[2-(2-pyridinyl)ethoxy]hexyl]-amino]methyl]benzenemethanol; diurectics, e.g. amiloride; anticholinergics e.g. ipratropium (e.g. as bromide), atropine, oxitrupium or tiotropium; hormones, e.g. cortisone, hydrocortisone or prednisolone; xanthines e.g. aminophylline, choline theophyllinate, lysine theophyllinate or theophylline; and therapeutic proteins and peptides e.g. insulin or glucagon. It will be clear to a person skilled in the art that, where appropriate, the therapeutic ingredients may be used in the form of salts, (e.g. as alkali metal or amine salts or as acid addition salts) or as esters (e.g. lower alkyl esters) or as solvates (e.g. hydrates) to optimise the activity and/or stability of the therapeutic ingredient and/or to minimise the solubility of the therapeutic ingredient in the propellant. It will be clear also that where appropriate, the therapeutic ingredients may be used in optically pure form.

The formulations according to the invention include those suitable for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the medicaments into association with the carrier which constitutes one or more accessory ingredients. In general the formulations are prepared by uniformly and intimately bringing into association the medicaments with liquid carriers or finely divided solid carriers or both and then, if necessary, shaping the product into the desired formulation.

Formulations for inhalation include powder compositions which will preferably contain lactose, and spray compositions which may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluoroethane, carbon dioxide or other suitable gas. Suitable aerosol formulations include those described in EP 0372777 and WO93/11743. Intranasal sprays may be formulated with aqueous or non-aqueous vehicles with the addition of agents such as thickening agents, buffer salts or acid or alkali to adjust the pH, isotonicity adjusting agents or anti-oxidants.

Capsules and cartridges or for example gelatin, or blisters of for example laminated aluminium foil, for use in an inhaler or insufflator may be formulated containing a powder mix of the medicaments and a suitable powder base such as lactose or starch. In this aspect, the medicaments are suitably micronised so as to permit inhalation of substantially all of the medicaments into the lungs upon administration of the dry powder formulation, thus the medicaments will have a particle size of less than 100 microns, desirably less than 20 microns, and preferably in the range 1 to 10 microns. Suitably, such particles of the medicaments may alternatively be produced by grinding in an air-jet mill, ball mill or vibrator mill, microprecipitation, spray-drying, lyophilisation, or recrystallisation from supercritical media.

Solutions for inhalation by nebulisation may be formulated with an aqueous and/or organic vehicle with the addition of agents such as acid or alkali, buffer salts, isotonicity adjusting agents or antimicrobials. They may be sterilised by filtration or heating in an autoclave, or presented as a non-sterile product. Suitable nebuliser devices for delivery of such formulations include pneumatic nebulisers and ultrasonic nebulisers as well as hand-held nebulisers (including the RESPIMAT™ device).

The use of aerosols for the administration of medicaments by peripheral aerosol pathways has been known for several decades. Such aerosols generally contain a medicament, one or more excipients such as solvents or surfactants and one or more propellants.

The most commonly used propellants in the past were chlorofluorocarbons such as CCl₃F (Freon® 11), CCl₂F₂ (Freon® 12), or CF₂ClCF₂Cl (Freon® 114). However, the recent phasing out of these propellant gasses due to their harmful effect on the ozone layer has caused manufacturers of aerosol sprays to use new propellant gases which protect stratospheric ozone. Such “ozone friendly” gases encompass hydrogen-containing fluorocarbons such as 1,1,1,2-tetrafluoroethane (CF₃CH₂F) and 1,1,1,2,3,3,3-heptafluoro-n-propane (CF₃CHFCF₃).

The replacement of the usual chlorofluorocarbon propellants by the “ozone friendly” propellants can be accompanied by problems of suspension stability and pharmaceutical performance through the life of the product. Various solutions to such problems have been described. For example, European patent application number EP0372777 discloses both suspension and solution formulations which comprise, in addition to the drug and propellant, a solvent and a surfactant as essential components of an aerosol formulation suitable for pharmaceutical use. International patent application numbers WO92/08446 and WO92/08447 disclose formulations of drugs such as salmeterol in which the medicament is coated with a surfactant. WO93/11743, WO93/11744 and WO93/11745 disclose suspension formulations of drugs including salmeterol which specifically exclude the presence of surfactant.

There is provided in one preferred aspect of the invention a pharmaceutical aerosol formulation consisting essentially of (or consisting of) R-salmeterol or a physiologically acceptable salt thereof and fluticasone propionate or a physiologically acceptable salt or solvate thereof, optionally one or more other therapeutic ingredients, and 1,1,1,2-tetrafluoroethane, 1,1,1,2,3,3,3-heptafluoro-n-propane or a mixture thereof as propellant.

The preferred aerosol formulations of the invention are essentially binary mixtures of medicaments and propellant and thus are substantially free of formulation excipients typically used in aerosol formulations such as surfactants and solvents. By “substantially free” is meant formulations which contain no significant amounts of excipients, i.e. less than the concentration of an excipient which would be required to have an effect on the characteristics of the formulation, for example less than 0.0001% w/w excipient based on weight of medicament.

The medicaments to be used in the inhaled formulations of the invention are in particulate form (for example micronised) and typically have a particle size such as to permit inhalation of substantially all of the medicament into the lungs upon administration of the aerosol formulation and will thus be less than 100 microns. desirably less than 20 microns, and preferably in the range 1 to 10 microns, for example, 1 to 5 microns.

The final aerosol formulation desirably contains 0.005-10% w/w, preferably 0.005-5% w/w, especially 0.01-1.0% w/w, of medicament relative to the total weight of the formulation. In one preferred aspect of the invention, the aerosol formulation contains 0.1-10% w/w of medicament relative to the total weight of the formulation.

The propellants for use in the preferred aerosol formulations of the invention are 1,1,1,2-tetrafluoroethane (CF₃CH₂F), 1,1,1,2,3,3,3-heptafluoro-n-propane (CF₃CHFCF₃) or mixtures thereof. The preferred propellant is 1,1,1,2-tetrafluoroethane.

The preferred aerosol formulations of the invention may be prepared by dispersal of the medicament in the selected propellant in an appropriate container, e.g. with the aid of sonication or a high shear mixer. The process is desirably carried our under anhydrous conditions to obviate any adverse effects of moisture on suspension stability.

The preferred aerosol formulations according to the invention form weakly flocculated suspensions on standing but which are easily redispersed by mild agitation to provide suspensions with improved delivery characteristics suitable for use in pressurised inhalers, even after prolonged storage. Avoiding the use of formulation excipients such as surfactants, solvents etc in the aerosol formulations according to the invention is also advantageous since the formulations may be substantially taste and odour free, less irritant and less toxic than conventional formulations.

The chemical and physical stability and the pharmaceutical acceptability of the aerosol formulations according to the invention may be determined by techniques well known to those skilled in the art. Thus, for example, the chemical stability of the components my be determined by HPLC assay, for example, after prolonged storage of the product. Physical stability data may be gained from other conventional analytical techniques such as, for example, by leak testing, by valve delivery assay (average shot weights per actuation), by does reproducibility assay (medicament per actuation), spray distribution analysis and focused beam reflectance.

The particle size distribution of the aerosol formulations according to the invention may be measured by conventional techniques, for example by cascade impaction (for example as defined in US Pharmacopoeia, 23/NF18 General Test <601>, pages 1762-1765) or by the “Twin Impinger” analytical process as defined in British Pharmacopaeia 1988, pages A204-207, Appendix XVII C. Such techniques enable the “respirable fraction” of the aerosol formulations to be calculated. As used herein reference to “respirable fraction” means the amount of medicament collected in the lower impingement chamber per actuation expressed as a percentage of the total amount of medicament delivered per actuation using the twin impinger method described above. The formulations according to the invention have been found to have a respirable fraction of 20% or more by weight of the medicament, preferably 25 to 70%, for example 30 to 60%.

The aerosol formulations according to the invention may be filled into canisters suitable for delivering pharmaceutical aerosol formulations. Canisters generally comprise a container capable of withstanding the vapour pressure of the propellant used such as a plastic or plastic-coated glass bottle or preferably a metal can, for example an aluminium can which may optionally be anodised, lacquer-coated and/or plastic-coated, which container is closed with a metering valve. Preferred metal canisters (for example those made of aluminium) for use with the formulations of the invention are described in European patent application number EP0642992 and International patent application number WO96/32150 (incorporated herein by reference). Such containers described therein comprise an internal coating of a pure fluorocarbon polymer such as PTFE or a blend of a fluorocarbon polymer and a non-fluorocarbon polymer such as a PTFE/PES polymer blend to minimise any deposition of the drug onto the canister wall.

The metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak pic, UK (e.g. BK300, BK356) and 3M-Neotechnic Ltd, UK (e.g. Spraymiser™).

Conventional bulk manufacturing methods and machinery well known to those skilled in the art of pharmaceutical aerosol manufacture may be employed for the preparation of large scale batches for the commercial production of filled canisters. Thus, for example, the canisters can be filled first with the powder and then with the propellant or alternatively filled with a prepared suspension of the powder in the propellant either as a single aliquot or as an aliquot of concentrated suspension followed by neat propellant to flush all the drug into the canister. A particularly preferred method is described and claimed (see claim 1 thereof in EP491261 incorporated herein by reference. This filling will preferably be carried out in a controlled atmosphere with a low relative humidity, in order to limit the effects of moisture on the drug particles during filling. Typically, in batches prepared for pharmaceutical use, each filled canister is check-weighed, coded with a batch number and packed into a tray for storage before release testing.

Each filled canister is conveniently fitted into a suitable channelling device prior to use to form a metered dose inhaler for administration of the medicament into the lungs or nasal cavity of a patient. Suitable channelling devices comprise for example a valve actuator and a cylindrical or cone-like passage through which medicament may be delivered from the filled canister via the metering valve to the nose or mouth of a patient e.g. a mouthpiece actuator. The metered dose inhaler may optionally comprise a dose counter for indicating the number of doses dispensed from or remaining in the canister. Metered dose inhalers are designed to deliver a fixed unit dosage of medicament per actuation or “puff”, for example in the range of 10 to 5000 microgram medicament per puff.

The filled canisters and metered dose inhalers described herein comprise further aspects of the present invention.

The following non-limitative Examples serve to illustrate the invention.

### A: METERED DOSE INHALER

### EXAMPLE 1

R-salmeterol xinafoate (5.8 mg) and fluticasone propionate (8.000 mg) are weighed directly into an 8 ml 0.6 mm walled aluminium canister coated internally with a PTFE/PES polymer blend as described in WO96/32150. A Valois DF60 metering valve is crimped into place then 1,1,1,2-tetrafluoroethane (to 6.000 g) added, then the filled canister is sonicated for five minutes. The resultant aerosol delivers 36.25 microgram R-salmeterol xinafoate and 50.0 mcg fluticasone propionate per actuation.

An alternative method for preparing the formulation described in Example 1 involves mixing the medicament and propellant in a pressure vessel. An aliquot of the resultant suspension, followed by an aliquot of propellant is filled into a closed canister via the metering valve.

Similar methods may be used for the formulation of Examples 2 and 3:

### EXAMPLE 2

| | Per actuation | |
|---|---|---|
| R-salmeterol | 36.25 | microgram |
| xinafoate | | |
| Fluticasone | 100 | microgram |
| propionate | | |
| 1,1,1,2- | to 37.50 | mg |
| Tetrafluoroethane | | |

### EXAMPLE 3

| | Per actuation | |
|---|---|---|
| R-salmeterol | 36.25 | microgram |
| xinafoate | | |
| fluticasone propionate | 250 | microgram |
| 1,1,1,2- | to 75.0 | mg |
| Tetrafluoroethane | | |

### B: DRY POWDER INHALERS

### EXAMPLE 4

| | Per blister | |
|---|---|---|
| R-salmeterol | 72.5 | microgram |
| xinafoate | | |
| fluticasone propionate | 250 | microgram |
| Lactose NF/BP | to 25.0 | mg |

The medicaments are micronised and bulk blended with the lactose in the proportions given above. The blend is filled into specifically constructed double foil blister packs to be administered by a Diskhaler (Trademark of Glaxo Group Limited).

Similar methods may be used for the formulations of Examples 5 to 7:

### EXAMPLE 5

| | Per blister | |
|---|---|---|
| R-salmeterol | 72.5 | microgram |
| xinafoate | | |
| fluticasone propionate | 200 | microgram |
| Lactose Ph. Eur. | to 25.0 | mg |

### EXAMPLE 6

| | Per blister | |
|---|---|---|
| R-salmeterol | 72.5 | microgram |
| xinafoate | | |
| fluticasone propionate | 500 | microgram |
| Lactose Ph. Eur. | to 25.0 | mg |

### EXAMPLE 7

| | Per blister | |
|---|---|---|
| R-salmeterol | 72.5 | microgram |
| xinafoate | | |
| fluticasone propionate | 100 | microgram |
| Lactose Ph. Eur. | to 25.0 | mg |

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A combination of R-salmeterol or a physiologically acceptable salt or solvate thereof and fluticasone propionate or a physiologically acceptable salt or solvate thereof.

2. A pharmaceutical formulation comprising R-salmeterol or a physiologically acceptable salt or solvate thereof and fluticasone propionate or a physiologically acceptable salt or solvate thereof, and a pharmaceutically acceptable carrier or excipient, and optionally one or more other therapeutic ingredients.

3. A pharmaceutical formulation comprising R-salmeterol xinafoate and fluticasone propionate, and a pharmaceutically acceptable carrier or excipient, and optionally one or more other therapeutic ingredients.

4. A pharmaceutical formulation according to claim 2 or 3 which is suitable for administration by inhalation.

5. A pharmaceutical aerosol formulation consisting essentially of R-salmeterol or a physiologically acceptable salt thereof, fluticasone propionate or a physiologically acceptable salt thereof, optionally one or more other therapeutic ingredients or physiologically acceptable salts or solvates thereof, and 1,1,1,2-tetrafluoroethane, 1,1,1,2,3,3,3-heptafluoro-n-propane or a mixture thereof as propellant.

6. A pharmaceutical aerosol formulation according to claim 5 wherein the R-salmeterol is in the form of its xinafoate salt.

7. A canister suitable for delivering a pharmaceutical aerosol formulation and capable of withstanding the vapour pressure of the propellant used, which is closed with a metering valve and contains a pharmaceutical formulation according to any one of claims 2 to 6.

8. A metered dose inhaler which comprises a canister according to claim 7 fitted into a suitable channelling device.

9. A method for the prophylaxis or treatment of a clinical condition in a mammal, such as a human, for which a selective β₂-adrenoreceptor agonist and/or anti-inflammatory corticosteroid is indicated, which comprises administration of a therapeutically effective amount of a pharmaceutical formulation according to any one of claims 2 to 6.

10. A method according to claim 9 wherein the clinical condition is a disease associated with reversible airways obstruction such as asthma, chronic obstructive pulmonary disease (COPD), respiratory tract infection or upper respiratory tract disease.
